(19) United States
(12) Patent Application Publication
(10) Pub. No.: US 2017/0100285 A1
(43) Pub. Date: Apr. 13, 2017

(54) PHOTOCOAGULATION WITH CLOSED-LOOP CONTROL

(71) Applicant: Novartis AG, Basel (CH)

(72) Inventors: Paul R. Hallen, Colleyville, TX (US); Michael J. Papac, North Tustin, CA (US); Hugang Ren, Irvine, CA (US); Lingfeng Yu, Rancho Santa Margarita, CA (US)

(21) Appl. No.: 14/880,435

(22) Filed: Oct. 12, 2015

(51) Int. Cl. A61F 9/008; A61B 5/1455

(52) U.S. Cl. CPC A61F 9/00823; A61B 5/14555; A61F 2009/00863; A61F 2009/00851; A61F 2009/00885; A61F 2009/00897

(57) ABSTRACT

A method for applying a photocoagulation procedure includes capturing a baseline image of a first location within a surgical site. The method further includes applying a photocoagulation treatment at the first location within the surgical site, the photocoagulation treatment using a laser tool having a set of operating parameters. The method further includes capturing an operation image of the first location within the surgical site at a point in time after the photocoagulation treatment has started. The method further includes, with a control system, performing a comparison of the operation image and the baseline image to determine whether a treatment objective has been achieved. The method further includes, with the control system, in response to determining that the treatment objective has not been achieved, adjusting the set of operating parameters to create a set of adjusted operating parameters and continuing the photocoagulation treatment with the set of adjusted operating parameters.

### TECHNICAL FIELD

The present disclosure is directed to methods and systems for ophthalmic medical procedures, and more particularly, to methods and systems involving control of photocoagulation procedures.

### BACKGROUND

Laser photocoagulation is a treatment procedure that is used for a variety of retinal diseases such as diabetic retinopathy (DR) and age-related macular degeneration (AMD). Laser photocoagulation can also be used to treat conditions such as retinal breaks or retinotomies. During a laser photocoagulation procedure, a short laser pulse is sent to the portion of the retina to be treated. The energy of the laser pulse is then absorbed by the retinal pigment epithelium (RPE). This causes a temperature increase within the neurosensory retina. The temperature increase to the retinal cells then produces structural changes of the neurosensory retina. Such structural changes can yield a therapeutic effect.

Generally, when a patient undergoes a photocoagulation procedure, the patient has a follow-up visit at some point in time after the procedure. During the visit, the patient's eye is examined to determine whether the photocoagulation procedure was successful. Various techniques can be used to examine the retina. In one example, an Optical Coherence Tomography (OCT) image of the treated area is captured. OCT imaging can allow for imaging tissue underneath the surface. Such imaging can be helpful when determining whether the photocoagulation treatment was successful.

If it is decided that further treatment is required, then the patient has to undergo an additional photocoagulation procedure. This can be time consuming and inefficient for both the patient and the surgical staff In addition, because multiple surgical procedures may be required, the treatment can become expensive for the patient. While such office based OCT can provide closed loop visualization, it does not provide closed loop control. Thus, there is a need for continued improvement in the use and operability of surgical systems and tools for various photocoagulation procedures.

### SUMMARY

According to one example, a method for applying a photocoagulation procedure includes capturing a baseline image of a first location within a surgical site. The method further includes applying a photocoagulation treatment at the first location within the surgical site, the photocoagulation treatment using a laser tool having a set of operating parameters. The method further includes capturing an operation image of the first location within the surgical site at a point in time after the photocoagulation treatment has started. The method further includes, with a control system, performing a comparison of the operation image and the baseline image to determine whether a treatment objective has been achieved. The method further includes, with the control system, in response to determining that the treatment objective has not been achieved, adjusting the set of operating parameters to create a set of adjusted operating parameters and continuing the photocoagulation treatment with the set of adjusted operating parameters.

According to one example, a control system for managing a photocoagulation procedure includes a processor and a memory comprising machine readable instructions that when executed by the processor, cause the system to receive a baseline Optical Coherence Tomography (OCT) image of a location within a surgical site within a patient's eye. The system is further to provide an initial set of operating parameters to a laser probe that is configured to perform a portion of a photocoagulation treatment and receive a live OCT image of the location. The system is further to analyze the baseline OCT image and the live OCT image to determine whether a treatment objective has been completed. The system is further to, in response to determining that a treatment objective has not been completed send an updated set of parameters to the laser probe. The system is further to, in response to determining that the treatment objective has been completed, save the updated set of parameters for use at a different location within the surgical site.

According to one example, a method for applying a photocoagulation procedure includes obtaining a baseline Optical Coherence Tomography (OCT) image of a first location within a surgical site, applying a photocoagulation treatment at the location within the surgical site, the photocoagulation treatment using a laser configured according to a set of parameters, and obtaining an operation OCT image of the location within the surgical site at a point in time after the photocoagulation treatment has started. The method further includes, with a computing system, performing a comparison of the operation image and the baseline image and in response to determining that a treatment objective has not been achieved, determining a set of adjusted parameters for the laser, the adjusted parameters being based the comparison. The method further includes continuing the photocoagulation treatment with the laser configured with the set of adjusted parameters.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is a diagram showing an illustrative ophthalmic operation system adjacent a surgical site such as an eye.

FIG. 2 is a flowchart showing an illustrative method for using live OCT imaging to provide closed-loop feedback during a photocoagulation procedure.

FIG. 3 is a flowchart showing an illustrative method using an OCT image after a period of treatment to provide closed-loop feedback during a photocoagulation procedure.

FIGS. 4A and 4B are diagrams showing a baseline image and an operation image respectively.

FIG. 5 is a diagram showing an illustrative scanning pattern that captures multiple treatment locations simultaneously.

FIG. 6A is a diagram showing an illustrative diagnostic image of an eye.

FIG. 6B is a diagram showing the diagnostic image overlaid with laser treatment locations.

FIG. 7 is a diagram showing treatment locations overlaid on a diagnostic image.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For simplicity, in some instances the same reference numbers are used throughout the drawings to refer to the same or like parts.

As described above, when a patient undergoes a photocoagulation procedure, the patient generally has a follow-up visit at some point in time after the procedure. During the visit, the patient's eye is examined to determine whether the photocoagulation procedure was successful. If it is decided that further treatment is required, then the patient may undergo an additional photocoagulation procedure, which can be undesirable for the patient.

According to principles described herein, images of the surgical site are obtained before the surgical procedure and live images are taken during the photocoagulation procedure. Using these images, feedback that indicates the efficacy of the photocoagulation procedure can be provided in real time. Using this feedback, various parameters associated with the laser that performs the photocoagulation process can be adjusted during the procedure to ensure that the photocoagulation process is performed as desired.

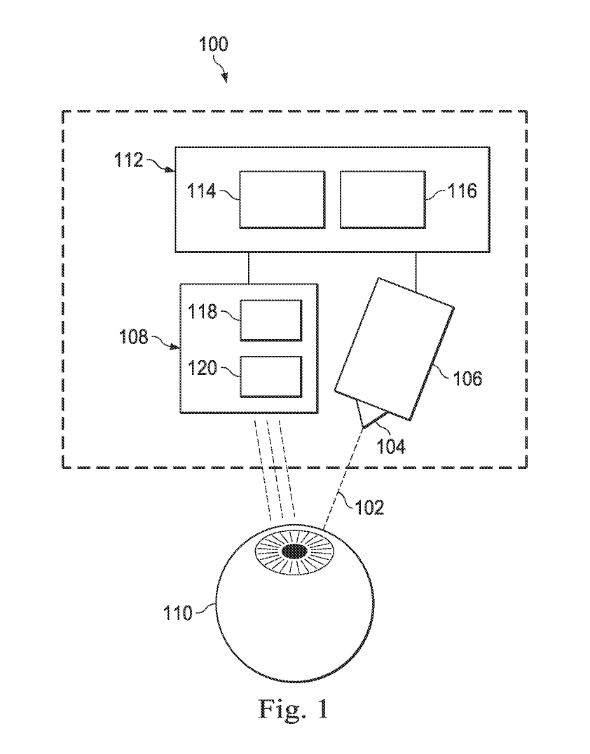

FIG. 1 is a diagram showing an illustrative ophthalmic operation system 100. According to the present example, the ophthalmic operation system 100 includes a photocoagulation system 106, an OCT imaging system 108, and a control system 112. The ophthalmic operation system 100 uses the photocoagulation system 106 to treat a region of the patient's eye 110. Such a region will be referred to as the treatment region. In some examples, the photocoagulation system is configured to direct a laser beam at multiple locations within a treatment region. Each location may be referred to as a treatment location.

The photocoagulation system 106 includes a laser tool 104 that emits a laser beam 102 at the treatment region. The photocoagulation system 106 may include the mechanical and electrical systems used to emit the laser beam 102 and redirect the laser beam 102 to various treatment locations within the treatment region. For example, the photocoagulation system 106 may include a set of movable mirrors that reflect the laser beam 102 to different locations within the treatment region based on the angular positions of such mirrors. The laser tool 104 includes a variety of adjustable operating parameters. Settings for these operating parameters may control the suitability and treatment capacities of the ophthalmic operation system 100. For example, the laser tool 104 may be set to generate the laser beam 102 at various power levels, frequencies, and other beam characteristics.

During surgery, the laser tool 104 is directed at a portion of the patient's eye that is to be treated (e.g., the treatment region) and the laser tool 104 is powered to treat tissue with the laser beam 102. At least some of the energy of the laser beam 102 is then absorbed by the retinal pigment epithelium (RPE). This results in a temperature increase within the neurosensory retina. The temperature increase causes structural changes to the neurosensory retina, which can yield a therapeutic effect. Based on various settings of the operating parameters of the laser tool 104, different structural changes can be achieved.

The OCT imaging system 108 obtains OCT images of the patient's eye 110. It uses various techniques to obtain depth resolved images of the patient's tissue beneath the surface of the tissue that are not able to be obtained from the use of a standard microscope. This is done using coherence gating based on light that is within the OCT spectrum. The OCT spectrum includes electromagnetic radiation having a wavelength between about 700 nanometers and 2600 nanometers, and in some cases can be extended to the visible light range of about 400 nanometers to 700 nanometers. By using coherence gating, the OCT imaging system 108 can display an image of tissue below the surface tissue and generate a cross-sectional view of such tissue. As such, the OCT imaging system 108 may be used to obtain a cross-sectional view of a specific location within the treatment region at which the laser tool 104 is directed. A benefit of this is that the OCT imaging system 108 can see how use of the photocoagulation system 106 affects the tissue below the surface of the treatment region.

The OCT imaging system 108 includes various components that are used to perform the OCT imaging function. For example, the OCT imaging system 108 may include an OCT light source 118 to project an OCT beam at the treatment region. The OCT imaging system 108 may also include an OCT capture device 120 that detects OCT light reflected from the treatment region. The OCT imaging system 108 then uses the information obtained by the OCT capture device 120 to construct an image of the treatment region. In some examples, the image may be a two-dimensional cross-section of the region of interest that provides a view beneath the surface of tissue within the treatment region. In some examples, the image may be a three-dimensional image that also provides a three-dimensional view beneath the surface.

The OCT imaging system 108 may be configured to take both still images and real-time images (e.g., live video) of the treatment region. As will be described in further detail below, the OCT imaging system 108 may take a still image of the treatment region before a photocoagulation process begins. This still image may be used as a baseline image. Then, during the photocoagulation process, the OCT imaging system 108 can obtain real-time images of the treatment region or additional still images. Images that are obtained during the photocoagulation procedure may be referred to as operation images.

The control system 112 is a computing system that may process images obtained from the OCT imaging system 108. For example, the control system 112 may obtain the baseline image and operation images from the OCT imaging system 108. Then, the control system 112 may perform various functions on or to such images to detect differences therebetween. The control system 112 may also perform functions to determine whether such differences represent or indicate that therapeutic objectives have been met or achieved. Based on whether such therapeutic objectives have been met or achieved, as determined based on the control system's 112 analysis of the images, the control system 112 may provide instructions to the photocoagulation system 106. Examples of instructions may be to stop the current procedure, to change the settings on the laser, or other instructions. The control system 112 can also determine which locations within a surgical site should be treated as will be described in further detail below.

The control system 112 also includes a processor 114 and a memory 116. The memory 116 may include various types of memory including volatile memory (such as Random Access Memory (RAM)) and non-volatile memory (such as solid state storage). The memory 116 may store computer readable instructions, that when executed by the processor 114, cause the control system 112 to perform various functions, including the analysis of the baseline and operation images as described herein. The memory 116 may also store data representing images captured by the OCT imaging system 108.

In some examples, the photocoagulation system 106 is designed to operate externally to the patient's eye. In such case, various lenses may be used to direct the laser beam 102 to the proper locations within the eye 110. In some examples, however, the photocoagulation system 106 may be configured as a probe that is designed to penetrate the patient's tissue. For example, the probe may be introduced into the patient through a cannula (not shown). Such a cannula may be inserted into the patient's eye 110 to allow the probe to pass therethrough. In some examples, the OCT imaging system 108 also may be integrated with such a probe. In some cases, however, the OCT imaging system 108 is external to the eye 110.

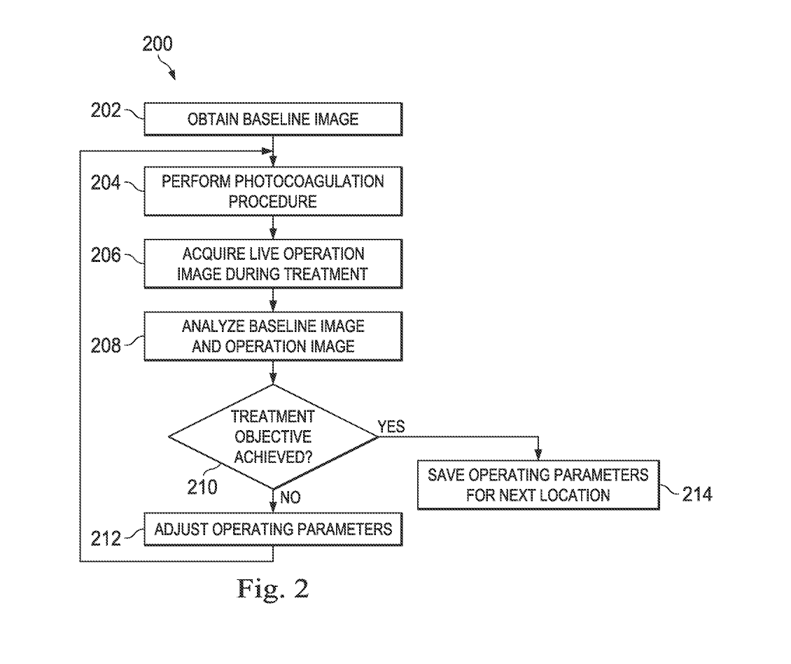

FIG. 2 is a flowchart showing an illustrative method for using live OCT imaging to provide closed-loop feedback during a photocoagulation procedure. While the following steps are described as being performed by a photocoagulation system (e.g. 106, FIG. 1), an OCT imaging system (e.g., 108, FIG. 1), and a control system (e.g. 112, FIG. 1), other systems or combination of systems may perform the steps.

According to the present example, at step 202, the OCT imaging system obtains a baseline image of a particular location within the treatment region. The baseline image may include a portion of the treatment region at which the laser beam will be directed. The baseline image serves as a reference image that can be compared to live images taken during the photocoagulation process.

At step 204, the photocoagulation system starts the photocoagulation process for the particular treatment location. This includes directing the laser beam at the particular treatment location within the treatment region. Various methods can be used to determine such treatment locations as will be described in further detail below. A variety of adjustable operating parameters that may be set in a manner that defines the nature of the treatment to be provided by the procedure.

In some examples, the operating parameters include the distance between a tip of the laser tool and the location within the region of interest being treated. In some situations it may be more beneficial to have the tip of the laser tool closer to the patient's eye and in some situations it may be more beneficial to have the tip of the laser tool farther from the patient's eye. In the case where the laser tool is external to the eye, the distance between the tip of the tool and the region of the eye being treated may be within a range of about 25 mm. Other ranges are contemplated as well. In some examples, the distance between the tip of the laser tool and the location within the region of interest being treated can be determined by analyzing the OCT image. For example, if the tip of the laser tool is close enough to the treatment region, the tip of the laser tool may be present within the OCT image. In such a case, analysis of the image can be used to determine the distance between the tip of the laser tool and the treatment region.

In some examples, the operating parameters include the laser power setting. The laser power setting corresponds to the energy output of the laser. In general, a higher powered laser will more quickly affect the tissue at which it is being directed. The laser power may be set within a range of about 250 mW. Other ranges are contemplated as well.

In some examples, the operating parameters include the laser spot size. When the laser beam is directed at tissue, it will produce a laser spot on that tissue. The laser spot size is a function of the beam characteristics as well as the distance between the tip of the laser tool and the tissue surface. The laser spot may be defined in a variety of ways. For example, the laser spot size may be defined by diameter of a circular spot size. The laser spot size may also be defined by area. In some cases, the laser spot size may be defined by depth or volume. In some examples, the spot size may be within a range of 100 μm to 500 μm.

In some examples, the operating parameters include the duration of a single laser pulse. The duration of the laser pulse may be based in part on or a function of the power setting of the laser as well as the distance between the tip of the laser tool and the tissue surface. Generally, a higher powered laser may be applied for a shorter duration. Conversely, a lower powered laser may be applied for a longer duration.

While in some cases, only a single pulse is fired for the treatment, in some examples, a series of smaller pulses may be applied. Thus, the operating parameters may include the characteristics of a series of pulses, such as pulse frequency and duty cycle. A duty cycle refers to the ratio of time the laser is on to the time the laser is off during each cycle.

In one example, the operating parameters include the frequency, and thus the wavelength, of the laser itself Lasers using different wavelengths of light may be used for different treatment options. In one example, the laser wavelength is within a range of 532 nm, 577 nm, 810 nm. Other ranges are contemplated as well. Other parameters, such as laser focal distance or gap between laser spots may be used as well.

At step 206, the OCT imaging system acquires a live operation image during treatment. The live operation image may be, for example, an OCT video stream of the treatment region that is captured during the photocoagulation procedure. Thus, as the procedure proceeds in real time, the live operation image will be updated in real time.

At step 208, the control system analyzes the baseline image and the operation image. Specifically, the control system compares the baseline image with the operation image to determine the difference therebetween. During the photocoagulation procedure, the tissue being treated undergoes structural changes. As will be described in further detail below, such structural changes may be reflected in the OCT image as changes in the intensity or polarization of the OCT image. For example, performing a laser photocoagulation treatment will typically cause scarring of the tissue underneath the surface. The size and nature of such scarring can be used to determine whether a treatment objective has been met. The scarring may be identified on an OCT image as a difference in image intensity between the baseline image and the operation image. Image intensity may correspond to brightness of the image, color of the image, or other indicators. Other factors may be used as well to determine the differences between the baseline image and the operation image in order to determine whether a treatment objective has been met.

At step 210, the control system determines whether the treatment objective has been achieved. The treatment objective may be defined by an operator and may be based on the condition being treated. Generally, the treatment objective is a specific amount of structural change to the tissue at which the laser beam is directed. For example, too little change may be less effective. For example, too little change may indicate that the tissue is not sufficiently treated. Conversely, too much change may cause issues. For example, too much change may indicate that the tissue has been over treated, potentially resulting in adverse side effects. If the treatment objective is to cauterize part of the retina, then the operator can define how much cauterization should take place as the treatment objective.

Determining whether the treatment objective has been achieved may be done in a variety of ways. In one example, it is known from previous experience that an ideal outcome will cause a certain amount of structural change that can be detected in the OCT image. Such structural changes may be reflected in the OCT image as a change of image intensity at the location within the treatment region that is being treated. Other changes within the OCT image can be used as indicia for determining whether the treatment objective has been achieved.

Various analytical tools may be used to help identify a satisfactory treatment objective for a particular surgical procedure. In one example, such tools may include an OCT angiography and an oximetry map of the surgical site. Such tools may provide information regarding how much structural change would be beneficial for a particular patient. Other tools for assisting with determining a treatment objective are contemplated.

If at step 210 it is determined that the treatment objective has been achieved, then it is known that the current settings of the operating parameters are sufficient to perform the desired procedure. Such operating parameters can then be saved for use with the next treatment location to be treated. Presumably the same laser settings that were effective at one treatment location will also be effective on another treatment location. But, if not, then the closed loop process illustrated in FIG. 2 will cause the operating parameters to be adjusted as will be described below.

If it is determined at step 210 that the treatment objective has not been achieved, then the control system adjusts the operating parameters at step 212. Specifically, the control system sends a revised set of operating parameters to the photocoagulation system. The method 200 then returns to step 204 at which the photocoagulation system continues the photocoagulation procedure. In some cases, the parameters are updated on a continuing basis. In other words, the parameters used by the laser may change in real time based on the treatment objective and the control system's analysis of the difference between the baseline image and the operation image.

The manner of adjustment of the operating parameters may be based on the difference between the live operation OCT image and the expected outcome. For example, if the affected area is too small, then the width of the laser beam can be increased. Conversely, if the affected area is too large, then the width of the laser beam can be reduced. If the desired structural changes have not yet been achieved, then the laser beam may be applied for a longer period of time or at a higher power level. Other changes as determined by the control system are contemplated.

In some cases, a human operator may manually make changes to the operating parameters. This may be done after the human operator views the baseline image and the live operation image. The human operator may use personal experience to determine a new set of operating parameters to help achieve the treatment objective.

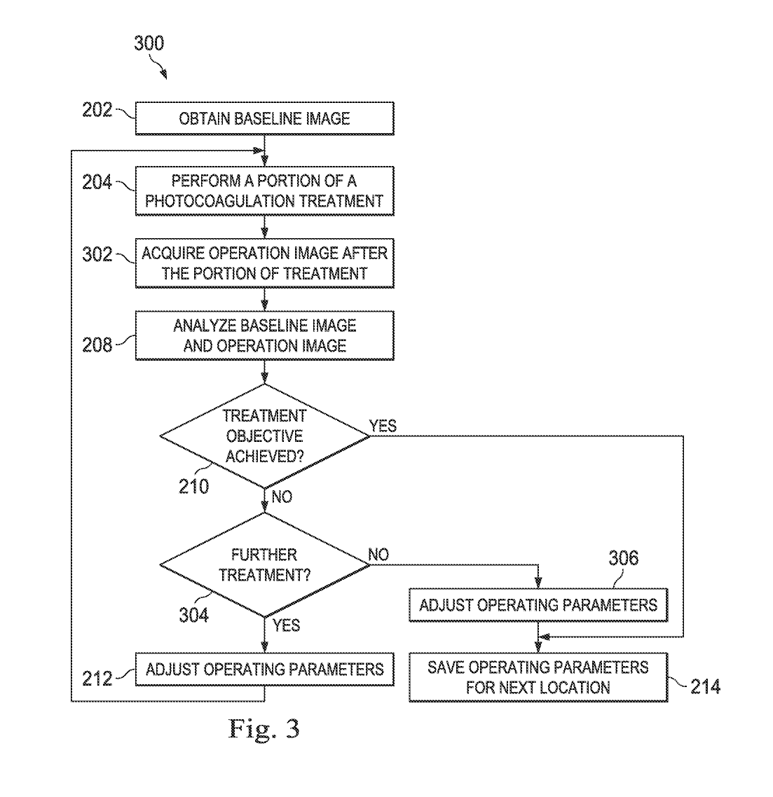

FIG. 3 is a flowchart showing an illustrative method using an OCT image after a period of treatment to provide closed-loop feedback during a photocoagulation procedure. The period of treatment corresponds to a portion of a procedure at one of several treatment locations that are treated during a single surgical visit. In some examples, instead of obtaining a live operation image during the photocoagulation procedure, a still image can be obtained after each laser firing. FIG. 3 repeats a number of the steps described with reference to FIG. 2. For the sake of discussion, these steps are given the same reference numerals in FIG. 3 that are used in FIG. 2. Further, the explanations and description given with reference to FIG. 2 is equally applicable to these steps in the context of FIG. 3.

According to the present example, the method 300 includes a step 202 for obtaining a baseline image and a step 204 for performing a first portion of the photocoagulation treatment. The treatment may be intended to only be a portion of the overall procedure. For example, the treatment may correspond to a single laser pulse.

At step 302, the OCT imaging system acquires an operation image after the first portion of the treatment. In this example, the operation image is a still image or non-live video image of the tissue under treatment. Then, at step 210, while the patient present, the control system determines whether the treatment objective has been achieved. If the treatment objective has been achieved, then the operating parameters used for the treatment are saved for use with the next treatment location to be treated during the present surgical visit.

If, however, it is determined that the treatment objective was not achieved, the method 300 proceeds to step 304. At step 304, the control system determines whether any further treatment should be performed. For example, if the treatment objective was not achieved due to excessive application of the laser, then further treatment may not be helpful. Thus, the method proceeds to step 306 at which the control system provides a revised set of operating parameters. For example, the revised set of operating parameters may be a lower power setting or shorter pulse duration. The revised parameters are then saved for the next location at step 214.

If the control system determines at step 304 that further treatment should be performed, then the control system adjusts the operating parameters at step 212. For example, if the treatment objective was not achieved due to the laser being under applied, then further treatments may allow the treatment objective to be reached. The method then returns to step 204 where an additional treatment is applied with the revised set of operating parameters.

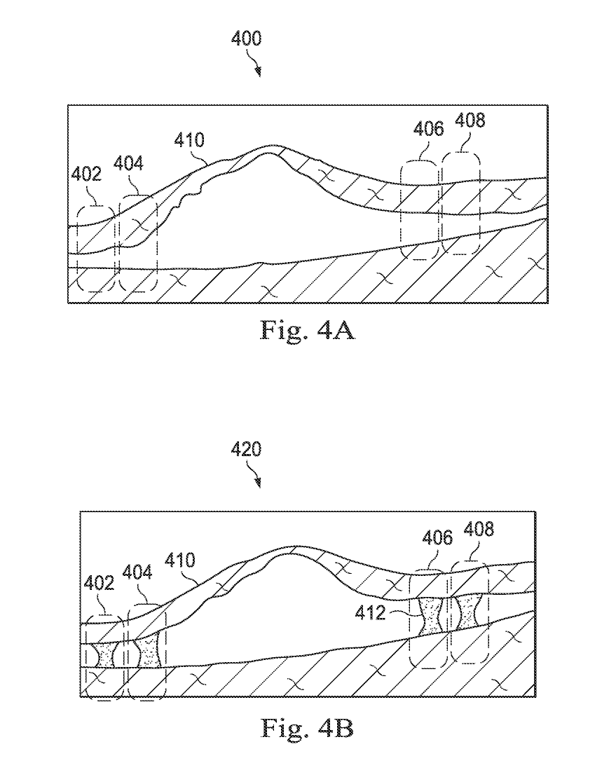

FIGS. 4A and 4B are diagrams showing an illustrated baseline image 400 and an illustrated operation image 420 respectfully. Both the baseline image 400 and the operation image 420 show a cross-sectional view of a treatment region. The surface 410 of the tissue can be seen in both images. In the baseline image 400, there are four locations 402, 404, 406, 408 that are to be treated with a photocoagulation treatment. Each location 402, 404, 406, 408 corresponds to a treatment location at which a laser beam is to be directed during treatment. FIGS. 4A and 4B are illustrated examples of an OCT image and do not necessarily represent how an OCT image actually appears.

The operation image 420 is taken at some point after the photocoagulation procedure has begun. In some cases, a laser that produces multiple beams at the same time is used to treat multiple locations simultaneously. Thus, all four locations 402, 404, 406, 408 may be treated at the same time. In some examples, however, the different locations are treated successively one at a time. As can be seen in FIG. 4B, scarring 412 appears underneath the surface 410 at the four locations 402, 404, 406, 408. Such scarring 412 may appear as an increase in image intensity at the corresponding regions. The nature of the change in image intensity may indicate that the treatment objective has been achieved.

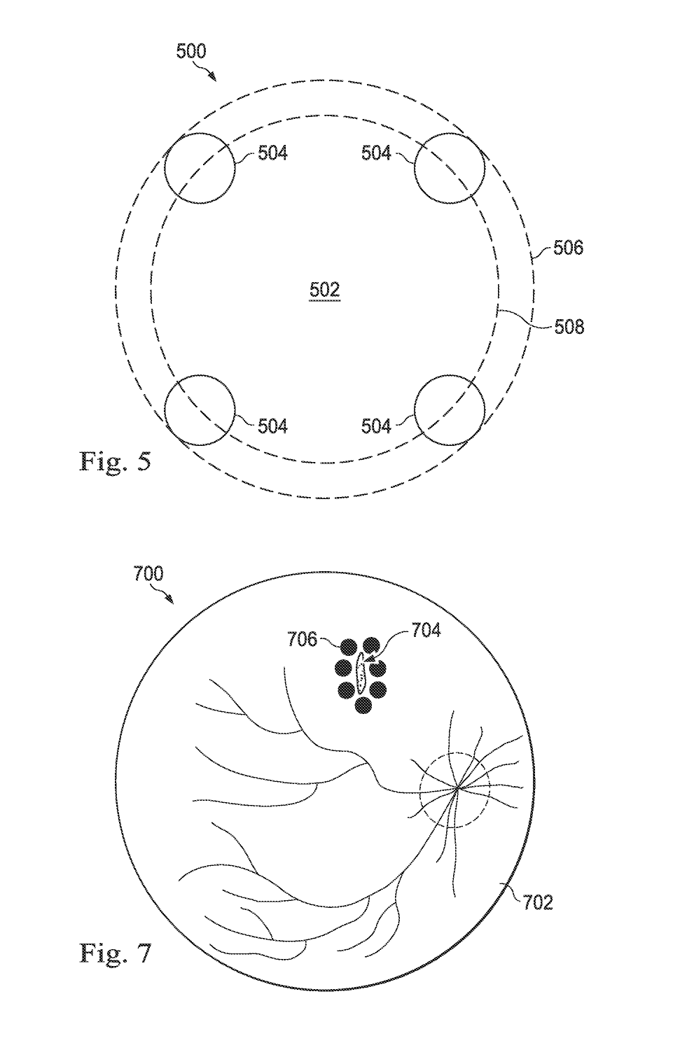

FIG. 5 is a diagram showing an illustrative scanning pattern that allows the OCT imaging system to capture multiple treatment locations simultaneously. As described above, some lasers are designed to produce multiple beams at the same time. Such a laser tool may be used to treat multiple treatment locations within a treatment region 502 at the same time. In the present example, four treatment locations 504 within a treatment region 502. The OCT imaging system (e.g. 108, FIG. 1) can be configured with a scanning pattern such that it captures an area 506 that encompasses the four treatment locations 504. In such a case, the OCT imaging system can obtain three dimensional images that include a three dimensional view beneath the surface. Thus, the OCT imaging system can obtain a baseline three-dimensional image for comparison with a live three-dimensional image.

In another example, the OCT imaging system can be configured with a circular or elliptical scanning pattern 508 such that the OCT imaging system scans across all treatment locations 504 as a cross-sectional scan. In such a case, the OCT imaging system can obtain cross-sectional images that include a view beneath the surface.

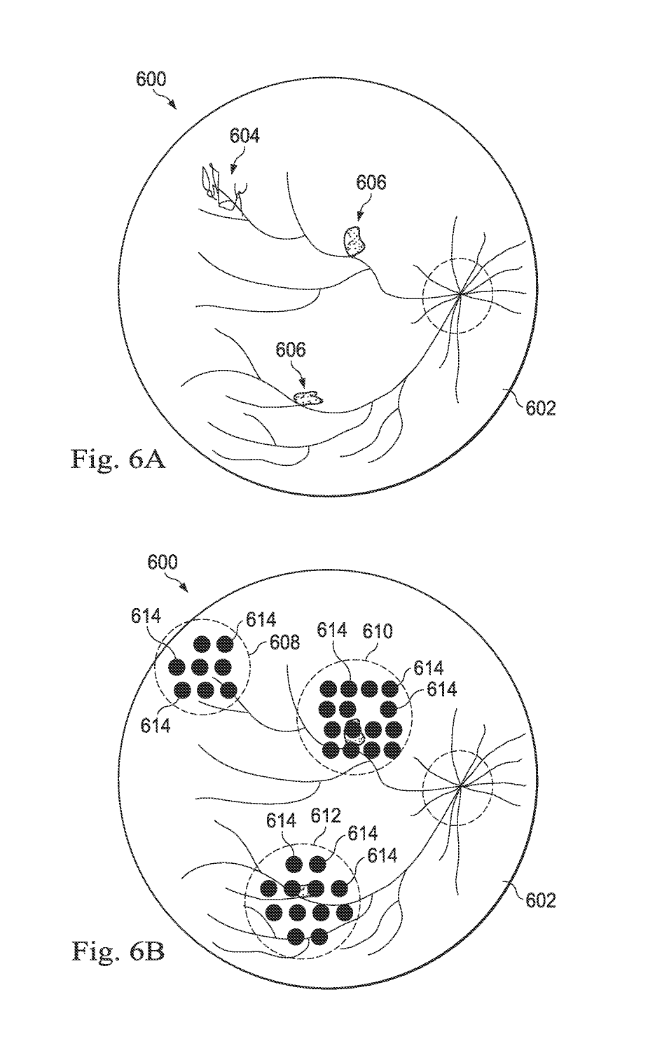

FIG. 6A is a diagram showing an illustrative diagnostic image 600 of a patient's retina 602. Before a laser photocoagulation procedure is performed, the treatment locations are identified. This may be done by obtaining the diagnostic image 600. In some examples, the diagnostic image 600 may be taken by a standard microscope imaging system. Such a microscope imaging system may be in communication with the control system (e.g. 112, FIG. 1).

The control system can be configured to analyze the diagnostic image 600 and automatically determine the treatment locations within the treatment region that should be treated. In the present example, the diagnostic image 600 indicates an abnormal section of vasculature 604 and some leaky vessels 606. Such conditions may be treated through photocoagulation processes. According to the present example, the control system can apply a function to determine the appropriate treatment locations for photocoagulation treatment. The control system may be configured to recognize certain conditions such as abnormal vasculature 604 or leaky vessels 606. Based on the size and shape of such conditions, the control system may determine the appropriate treatment locations.

FIG. 6B is a diagram showing the diagnostic image 600 overlaid with treatment locations 614 to be treated with a laser as described above in FIG. 2 and FIG. 3. Each treatment location 614 corresponds to a point at which a single laser beam is to be projected. According to the present example, after the control system applies the function to determine the treatment location, there are three sets 608, 610, 612 of treatment locations. The first treatment location set 608 corresponds to the abnormal vasculature 604. The second treatment location set 610 corresponds to one of the leaky vessels 606. The third treatment location set 612 corresponds to the other leaky vessel 606.

In some examples, a scanning pattern can be designed to capture an entire treatment location set 608, 610, 612. For example, when treating the abnormal vasculature 604, the OCT imaging system can use a scanning pattern that covers every location 614 within the first treatment location set 608. Likewise, when treating either of the leaky vessels 606, the OCT imaging system can use a scanning pattern that encompasses the respective treatment location set 610, 612.

FIG. 7 is a diagram showing treatment locations overlaid on a diagnostic image. Conditions other than leaky vessels and abnormal vasculature can be treated using a photocoagulation procedure. For example, retinotomies and retinal breaks can be treated as well. FIG. 7 illustrates a diagnostic image 700 of a retina 702 with a retinal break 704. FIG. 7 also illustrates the treatment locations 706 used to treat the retinal break 704. The positions of the treatment locations 706 are positioned around the external edges of the retinal break 704. Other patterns for treating such retinal breaks may be used as well.

Using principles described herein, photocoagulation treatment for a variety of diseases and conditions can be achieved more efficiently. Such diseases and conditions include, but are not limited to, a retinal break, a retinotomy, a choroid neovascularization (CNV), diabetic retinopathy, and macular degeneration. More specifically, because real time feedback is provided as to the status of the procedure and the effects thereof, the treatment can be adapted in real time to achieve the desired outcome.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

1. A method for applying a photocoagulation procedure, the method comprising:
capturing a baseline image of a first location within a surgical site;
applying a photocoagulation treatment at the first location within the surgical site, the photocoagulation treatment using a laser tool having a set of operating parameters;
capturing an operation image of the first location within the surgical site at a point in time after the photocoagulation treatment has started;
with a control system, performing a comparison of the operation image and the baseline image to determine whether a treatment objective has been achieved; and
with the control system, in response to determining that the treatment objective has not been achieved, adjusting the set of operating parameters to create a set of adjusted operating parameters and continuing the photocoagulation treatment with the set of adjusted operating parameters.

2. The method of claim 1, further comprising, in response to determining that the treatment objective has been achieved;
saving the adjusted set of operating parameters; and
applying the photocoagulation treatment to a second location within the surgical site with the laser tool using the set of adjusted parameters.

3. The method of claim 1, wherein the set of operating parameters includes at least one of: laser power, laser duty cycle, laser spot diameter, gap between each laser spot, laser spot depth, laser spot volume area, laser pulse duration, laser frequency, laser focal distance, and distance between a laser probe and the first location.

4. The method of claim 1, wherein the operation image is a live image of the surgical site.

5. The method of claim 1, wherein the operation image is a still image that is captured after a set period of laser application.

6. The method of claim 1, wherein the baseline image and the operation image comprise Optical Coherence Tomography (OCT) images.

7. The method of claim 1, further comprising, while applying the photocoagulation treatment to the first location within the surgical site, using a multi-beam laser to perform the photocoagulation treatment to additional locations within the surgical site.

8. The method of claim 7, wherein the operation image includes the first location and the additional locations within the surgical site.

9. The method of claim 1, further comprising, determining the treatment objective based in part on at least one of: an OCT angiography and an oximetry map of the surgical site.

10. The method of claim 1, wherein the first location within the surgical site is automatically determined based on an analysis of a diagnostic image of the surgical site.

11. The method of claim 1, wherein the laser is integrated with a probe that is configured to be inserted into a patient's eye through a cannula.

12. The method of claim 11, wherein an imaging system that captures the operation image is integrated with the probe.

13. The method of claim 1, wherein the laser is external to a patient's eye.

14. The method of claim 1, wherein an imaging system that captures the operation image is external to a patient's eye.

15. A control system for managing a photocoagulation procedure, the system comprising:
a processor; and
a memory comprising machine readable instructions that when executed by the processor, cause the system to:
receive a baseline Optical Coherence Tomography (OCT) image of a location within a surgical site within a patient's eye;
provide an initial set of operating parameters to a laser probe that is configured to perform a portion of a photocoagulation treatment;
receive a live OCT image of the location;
analyze the baseline OCT image and the live OCT image to determine whether a treatment objective has been completed;
in response to determining that a treatment objective has not been completed, send an updated set of parameters to the laser probe; and
in response to determining that the treatment objective has been completed, save the updated set of parameters for use at a different location within the surgical site.

16. The system of claim 15, wherein determining whether the treatment objective has been completed comprises determining a size of scarring formed underneath a surface of tissue within the surgical site.

17. The system of claim 16, wherein the size of the scarring is determined at least in part based on intensity or polarization property differences between the baseline OCT image and the live OCT image at the location within the surgical site.

18. A method for applying a photocoagulation procedure, the method comprising:
obtaining a baseline Optical Coherence Tomography (OCT) image of a first location within a surgical site;
applying a photocoagulation treatment at the location within the surgical site, the photocoagulation treatment using a laser tool configured according to a set of parameters;
obtaining an operation OCT image of the location within the surgical site at a point in time after the photocoagulation treatment has started;
with a computing system, performing a comparison of the operation image and the baseline image; and
in response to determining that a treatment objective has not been achieved, determining a set of adjusted parameters for the laser tool, the adjusted parameters being based the comparison; and
continuing the photocoagulation treatment with the laser tool configured with the set of adjusted parameters.

19. The method of claim 18, wherein the photocoagulation treatment comprises treatment for at least one of: a retinal break, a retinotomy, a choroid neovascularization (CNV), diabetic retinopathy, and macular degeneration.

20. The method of claim 18, wherein the operation OCT image is obtained using an OCT scanning pattern designed to encompass or cross multiple treatment locations.

21. The method of claim 18, further comprising:
determining a distance from a tip of the laser tool and the first location by analysis of an OCT image that contains both the tip of the laser tool and the first location;
and providing the distance to a user.
